(19) United States
(12) Patent Application Publication
(10) Pub. No.: US 2022/0287637 A1
(43) Pub. Date: Sep. 15, 2022

(54) APPARATUS FOR PROCESSING SENSOR SIGNALS

(71) Applicant: CHRONOLIFE SAS, Paris (FR)

(72) Inventors: Guillaume Chenegros, Bassou (FR); Harmony Roger, Chevry-Cossigny (FR); Pascal Dumas, Versailles (FR)

(21) Appl. No.: 17/634,310

(22) PCT Filed: Jul. 29, 2020
(86) PCT No.: PCT/EP2020/071391

§ 371 Date: Feb. 10, 2022

(30) Foreign Application Priority Data
Aug. 12, 2019 (EP) PCT/EP2019/071632

(51) Int. Cl. A61B 5/00

(52) U.S. Cl. CPC A61B 5/6804; A61B 5/0017; A61B 2562/166; A61B 2560/0214; A61B 2562/18

(57) ABSTRACT

Electronic devices embedded in textile materials provide an improved connection of the electronic device with a textile without impairing the movement of a user. An apparatus for processing sensor signals includes an electronic device to process user biometric signals and a plurality of electrically conducting pads. Each pad is connected to an electrically conducting wire contacting a sensor corresponding to a user anatomic region and providing a sensor signal to the electronic device. A textile interface for a wearable fabric has a first and second surface and a porosity providing a fluidic communication between the surfaces. The electronic device, the pads, and the conducting wires are arranged on the first surface and attached to the textile interface. A coating material covers the electronic device and the pads and extends from the first to the second surface so as to fluidically seal the electronic device and the pads.

### CROSS-REFERENCE TO RELATED APPLICATIONS

This application is the U.S. National Stage of PCT/EP2020/071391 filed Jul. 29, 2020, which claims priority to European PCT Patent Application No. PCT/EP2019/071632 filed Aug. 12, 2019, the content of both of which are incorporated herein by reference in their entirety.

### FIELD OF THE INVENTION

The invention relates to the field of electronic devices embedded in textile materials, in particular a piece of clothing with an embedded apparatus for processing signals as well as methods for producing such an apparatus.

### BACKGROUND OF THE INVENTION

Electronic devices for detecting and processing biometric signals of a user generally require that one or more detection units, or sensors, are in proximity of or even in direct contact with an anatomic region of a user. For example, one or more sensors may be applied to the skin of a user so as to perform a temperature measurement or determine physiological parameters. For example, a plurality of sensors may be applied to the chest area of a user so as to detect electrical signals related to a cardiac function of the user. Monitoring the various signals delivered by these sensors allows determining a user specific physiological condition that might be impaired. For example, when an individual is having a seizure, specific signal features appear on the signals corresponding to the electrocardiogram (ECG) or to respiration. To receive and process the signals and to determine such biometric parameters, the sensors are connected by a plurality of wires to an electronic device, which may be fixed to a garment or clothing item using e.g. one or more straps or via integrated push buttons.

However, the use of electronic devices in garments poses various problems. For example, external influences due to changing weather conditions or transpiration of the user may result in liquids accumulating at the electronic components, which may cause corrosion or a short-circuiting, eventually leading to a malfunctioning of the electronic device. To overcome such liquid penetration, a casing may be provided for one or more components, which may reduce or avoid such undesirable effects, but results in a bulky device and impairs the free movement of the user. Furthermore, the electronic device needs to be removed from the garment to allow the garment to be washed and to enable a charging or removal of the batteries of the electronic device, which may require the removal of the casing from the electronic device. Such removal may be impractical and may be accidentally forgotten prior to washing, e.g. in the case wherein the electronic device has relatively small dimensions, thereby resulting in a loss of functionality.

In addition, the wiring of the sensors may not be sufficiently held in place on the anatomic region of the user, which may be caused by the movement and/or the transpiration of the user. By the same token, the connecting region of the wires and the electronic device may be loosened due to the movement of the anatomic region, e.g. when a user is exercising, resulting in a loss of sensory signals and an incomplete determining of the corresponding biometric parameter.

Accordingly, there is a need for electronic devices and methods adapted for integration of the electronic device into a fabric and which reduce the above problems and which provide an improved connection of the electronic device with a textile without impairing the movement of a user.

### SUMMARY OF THE INVENTION

It is an object of the present invention to provide an apparatus for processing sensor signals having an electronic device and a textile interface which abrogates at least some of the above observations being undesirable for the user or implementation in a garment.

Accordingly, in a first aspect, an apparatus for processing sensor signals is suggested, which comprises an electronic device configured to process sensor signals of a user and a plurality of electrically conducting pads connected to the electronic device, wherein each pad is connected to a respective electrically conducting wire configured for contacting a sensor corresponding to an anatomic region of a user and for providing a sensor signal to the electronic device. According to a preferred embodiment, the pad is connected to a respective electrically conducting wire by sewing or similar means. According to preferred embodiment, this sewing is performed with the same conducting wire used for contacting one of the said sensors.

The apparatus furthermore comprises a textile interface for use in a wearable fabric, which comprises a first surface and a second surface and has a porosity providing a fluidic communication between the first surface and the second surface. The electronic device, the plurality of pads, and the plurality of conducting wires are arranged on the first surface of the textile interface and are attached to the textile interface. It should be noticed that according to a preferred embodiment, the conducting wires are sewn over the said textile interface, therefore each stitch might be present on both the first surface and the second surface.

The apparatus further comprises a coating material, which covers at least the electronic device and the plurality of pads and extends from the first surface to the second surface so as to fluidically seal the electronic device and the plurality of pads.

The electronic device may be any device capable of processing sensor signals related to biometric parameters. For example, the electronic device may be a medical device configured to detect or record e.g. electrocardiogram (ECG), electroencephalogram (EEG), breathing pattern (including abdominal and costal patterns), lung impedance, body temperature, muscle activity or contraction, blood pressure and any other physiology signal. Accordingly, the electronic device may be part of a medical interface that enables patient measurements over prolonged time periods, e.g. for monitoring purposes or detection of physiological problems. By providing the conducting wires directly on the textile interface that may be integrated or embedded in a wearable fabric, a correct placement and contact with the appropriate sensor corresponding to an anatomic region may be ensured.

By the same token, the electronic device may also be configured as a wearable device to be worn during leisure or while performing sports. The electronic device may e.g. record particular movements and/or may record physiological parameters while exercising so as to evaluate the performance or monitor the user, e.g. to ensure that the user is exercising within predefined physiological boundaries.

According to preferred embodiment, the electronic device may also comprise an element for providing a required electrical energy e.g. a battery, preferably a welded battery.

In order to provide said measurements, the conducting wire may be configured to be coupled at a free end with a respective sensor and to receive and forward an electronic or optoelectronic signal, which may hence be received by a corresponding conducting pad. Accordingly, each conducting wire may be comprised of an electrically conducting material such as a metal, preferably a corrosive resistant metal. According to preferred embodiment, said conducting wire is comprised of fiber material (e.g. yarn), in particular a textile fiber, which is conductive. It generally comprises a base layer composed of a synthetic (e.g. polyamide) and/or natural fiber (e.g. cotton) combined with a conductive element formed, for example, of a plurality of nanoparticles or a permanently bonded silver layer. Ideally the selected conductive wire retains the flexibility and durability of the textile material. The conducting wire may have various dimensions and thicknesses so as to accommodate for different anatomical regions and biometric functions or to accommodate a sewing processing step. The number of conducting wires and conducting pads may vary depending on the required number of sensors and may range between 2 and 20, preferably between 5 and 13, to provide a number of accurate measurements while maintaining an average complexity of the control logic and while keeping the overall dimensions small.

The communication may either be one directional or bi-directional, such that the electronic device may only receive sensor signals or may be configured to both receive and output signals from and to the user, respectively. The electronic device comprises a control logic, e.g. in the form of a control unit and/or evaluation unit, which is connected to the conducting wires via the respective conducting pads, wherein the conducting pads may also be connected to each other.

Attachment of the various components to the textile interface may be provided e.g. by means of a preliminary or primary attachment with a fabric material or other material so as to hold the components at their respective predefined position on the textile interface. According to a preferred embodiment, the electronic device is attached to the textile interface at the conducting pad by sewing or similar means with conducting wire, preferably with electrically conducting yarn. According to a preferred embodiment, this sewing is performed with the same conducting wire or conductive yarn used for contacting with the sensors.

The final attachment may then be achieved by means of the coating material, which penetrates the textile interface and provides a continuous layer on both the second surface and the first surface of the textile interface and covering the electronic device, the battery when present, and the conducting pads. Thereby, the coating material provides an encapsulation of the electronic device, the battery when present, and conducting pads, which provides a material bonding or linkage and securely fixes the components to the textile interface. By the same token, the conducting wires may at least in part be covered with the coating material, e.g. at a connecting region extending from the conducting pads.

The coating has the advantage that a cover may be omitted and the thickness of the apparatus may be reduced, such that the thickness and hence bulkiness of the apparatus may be reduced and the apparatus may be easily implemented in a wearable fabric without impairing the movement of a user. Furthermore, the coating ensures that liquids may not penetrate the coating material, such that corrosion of the inner electronic components of the apparatus may be avoided. In addition, the direct coating increases the robustness by preventing a relative movement of the electronic components and by increasing the force distribution upon accidental impact. Thereby, the apparatus may also be machine washed without requiring a dismounting of particular components, such that a garment with an implemented apparatus does not require particular attention, which makes the product as a whole much more user friendly.

The coating material is hence a material that is not water-soluble and prevents penetration of liquids, e.g. water, so as to fluidically seal the electronic components. Furthermore, the coating material is preferably a non-conducting material to provide an electric isolation and to avoid the necessity of providing a primary coating with an electrically non-conducting material.

For easy implementation of the apparatus into a garment, the textile interface is preferably a flexible and/or resilient and/or elastic material, e.g. an elastomer or a poly(organo)siloxane (silicone) This not only provides that the textile is easy to handle, but may also conform to an anatomic region of a user. Accordingly, the borders or boundaries of the textile interface may e.g. be sewn into a wearable fabric and may be easily folded into the seams of a garment prior to finishing the garment. Thereby, the apparatus may be fully embedded into the wearable fabric and may hence not be visible to a user, thereby increasing the acceptance of the garment to a user.

In addition to the requirement of fluidically sealing the electronic components, and the battery when present, the coating material is preferably a flexible material, such that any movement of the apparatus is not significantly impaired by the coating or may result in a rupture of the continuous coating. The flexibility of the material may furthermore be experienced as more comfortable, when the apparatus is implemented in a clothing item and may provide a soft touch, preferably providing a resilience and low roughness. Preferably, the coating material comprises silicone or essentially consists of a silicone-based material. Such material allows a cost-effective encapsulation of the electronic components, a fast curing and an easy to handle processing while comprising a sufficient structural integrity and robustness for implementation in a clothing item and allowing, e.g., a machine washing.

The coating material may also comprise more than one layer, wherein each layer has been sequentially added after curing of the previous layer. The multiple layers may be based on the same material or may differ in their properties, wherein at least one layer ensures a fluidic sealing of the respective electronic components. The use of more than one layer may facilitate the initial adherence of the coating material to the respective electronic components and the textile interface, such that a primary fixation is established. Accordingly, this may facilitate the handling and processing of the apparatus and allows further layers to be added at a different time point and/or positioning of the apparatus.

Preferably, the porosity of the textile interface is provided by a plurality of holes at essentially equal spacing from each other. In other words, the textile interface may comprise an essentially homogeneous first and second surface. Preferably, the textile interface is a mesh material, most preferably tulle or a tulle-like material. The provision of the plurality of holes not only ensures that the coating material may easily penetrate the first and second surface and facilitate the distribution of the coating material, e.g. when the coating material is poured or sprayed onto the respective components of the apparatus, but also facilitates the implementation of the apparatus into a clothing item by ensuring that fixation may be provided at every region of the textile interface due to the homogenous distribution of the plurality of holes.

For example, the textile interface may simply be sewn into the seams of a clothing item or garment by means of the holes without impairing the structural integrity of the textile interface. At the same time, the plurality of holes ensures a full encapsulation of the respective electronic components. Thereby, the apparatus may be easily integrated or embedded into the clothing item while the respective electronic components are being protected from external influences. The use of a mesh or tulle material furthermore increases the breathability of the apparatus, such that the apparatus may also be implemented in e.g. sport gear or leisure equipment while at the same time reducing the overall weight of the apparatus.

Advantageously, each conducting wire is sewn into the textile interface, wherein the thread is the conducting wire and traverses the first and second surface of the textile interface. The attachment of the conducting wire may hence be primarily achieved by the sewing arrangement of the conducting wire. In order to provide such arrangement, the conducting wire may comprise e.g. a flexible material that allows bending and folding so as to orientate the conducting wire in various directions and to provide a sewing pattern for sufficient structural stability. For example, the conducting wire may comprise of one or more thin metal fibers that are sewn into the textile interface using conventional patterns and which together define the conducting wire. According to preferred embodiment, said conducting wire comprises conductive yarn that are sewn into the textile interface using conventional patterns and which together define the conducting wire. According to a preferred embodiment, said conductive yarn comprises metallic nanoparticles or metallic nanoparticles permanently bonded or plated metallic particles. According to a further preferred embodiment, said metallic particles or nanoparticles are silver particles.

The sewing engagement of the conducting wire with the textile interface hence achieves that the conducting wires are securely attached to the textile interface and do not require additional attachment means such as, e.g., mechanical fasteners or positive locking interfaces. To facilitate such sewing arrangement, the textile interface is preferably formed as a mesh or tulle material, such that the sewing does not significantly affect the structural stability of the textile interface and no additional holes have to be created in the textile interface.

As described in the above, the use of a mesh or tulle material furthermore has the advantage that the coating material may easily penetrate the first and second surface and facilitate the distribution of the coating material, e.g. when the coating material is poured or sprayed onto the respective components of the apparatus. Thereby, the covering of the conducting wires on each surface of the textile interface is facilitated. This is particularly the case when using a coating material that is flexible, such as silicone or a silicon-based material, which e.g. may be provided at a predefined viscosity during application to facilitate both the spreading and the adhesion of the coating material on both of the surfaces and the conducting wires. In addition, the use of such coating material may improve the haptics or tactile experience on both surfaces, also when the conducting wires engage the textile interface and traverse the first and second surface of the textile interface, since these materials may provide a soft touch and low roughness.

In addition, such fixation also provides that the conducting wires may be held at a predefined region, such that each conducting wire may receive electrical signals of said region via a corresponding sensor and the measurement accuracy and/or validity may be increased. In addition, mismatch or accidental misplacement of the conducting wires is avoided and user considerations, actions, and cognitive efforts may be reduced or are no longer required.

The arrangement of the conducting wires may depend on the type and the area for which measurements are to be obtained. Accordingly, the length and thickness of the conducting wires may be varied and the spacing between the conducting wires at the conducting pads, i.e. at the connecting region, may be essentially equal or may vary depending e.g. on the geometry and circuitry of the control logic of the electronic device.

Furthermore, each conducting pad may be secured to the textile interface by the plurality of conducting wires. For example, the conducting wires that are used as a thread may cover a portion of the electronic pads, e.g. one or more corners or edges of the respective electronic pad. This not only improves the connection between the respective conducting wire and electronic pad, but also increases the signal transduction due to a larger contacting surface.

To further improve the fixation of the conducting pads, each conducting pad preferably comprises at least one through hole and is sewn into the textile interface by the respective conducting wire extending through the at least one through hole. Thereby, each conducting wire may be connected to the respective conducting pad by at least one full loop, which secures the connection both between the conducting wire and the conducting pad and between the conducting pad and the textile interface. Furthermore, this avoids any relative movement between the conducting wires and the conducting pads.

The mechanical fixation of the conducting pads to the textile interface and the mechanical linkage between each conducting pad and the respective conducting wire may be further improved by providing additional holes in each conducting pad. Accordingly, each conducting pad preferably comprises two or three through holes. Thereby a more symmetrical and more profound fixation may be achieved, wherein a tilting of the respective conducting pad may be avoided and proper attachment of the connected conducting wire is hence also ensured during deformation of the apparatus, e.g. due to movement, for example when the apparatus is implemented in a clothing item.

Since the apparatus may be integrated in a clothing item, the apparatus may be sized and dimensioned accordingly. For example, it may be required that a height or thickness of the apparatus is kept small, so as to provide an essentially flat apparatus. To achieve such reduced height, each conducting wire may extend from the respective pad in a plane defined by the electronic device and the plurality of conducting pads. Accordingly, the conducting wires may have various orientations and lengths yet essentially remain in the same plane as the electronic device and conducting pads. In other words, the conducting wires may extend along the first and second surface of the textile interface yet do not extend in a direction perpendicular to said surfaces so as to avoid an increase in thickness of the apparatus.

The conducting wires may also be formed so as to accommodate or adapt to a variety of anatomic regions and movements of a user, which may not only have different curvatures, but also differ in the relative movement of the respective body part. Therefore, each conducting wire advantageously comprises an essentially straight portion, a curved portion, and/or a bent portion. For example, a straight portion may be advantageous in a situation where a straight movement is to be expected whereas a bent or curved portion may be advantageous in cases of an expected relative movement or bending of connected body parts or when an anatomic region comprises a curvature itself. Furthermore, bent or curved portions facilitate the detection of biometric signals at predefined positions without requiring a modification of the positioning of the corresponding conducting pads, such that the area of the apparatus that is provided with the coating material may be reduced.

Although a variety of conducting linkages may be provided between each conducting wire and the respective conducting pad, such linkage is preferably achieved by the conducting wire itself. Therefore, to ensure a full encapsulation of the conducting pads and the electronic device, and the battery when present, the coating material preferably covers a portion of each conducting wire connecting the conducting wire to the conducting pad. Thereby, a direct linkage is provided while at the same time a compact and fluidically secured configuration is achieved.

A compact design of the apparatus may also be achieved by using conducting pads that are integrated on the electronic device. For example, the electronic device may comprise a circuitry in the form of an electronic board or card, wherein the conducting pads are arranged at a predefined region. Thereby, the conducting pads are within the same plane as the electronic device, such that a thickness of the apparatus may be kept small. Furthermore, integration on the electronic device increases the structural integrity of the apparatus and reduces the occurrence of contact loss between the circuitry and the conducting pads.

Alternatively, the conducting pads may also be in direct contact with the textile interface and/or may be spaced apart from the electronic device. This results in an even more versatile arrangement of the electronic device and the conducting pads and further reduces the rigidity or bulkiness of the apparatus, allowing the apparatus to be more flexible, which may be advantageous when implementing the apparatus in a clothing item. Since the conducting pads are furthermore arranged on the same surface of the textile interface, said conducting pads are essentially also in the same plane as the electronic device. In such arrangement, the conducting pads may at least partially form an integrated circuit by means of connecting conducting wires, thereby forming a control logic, such that the electronic device may at least partially be substituted by the conducting pads and conducting wires. In other words, such arrangement may provide a “smart” textile and, depending on the configuration, may even fully omit the requirement of a separate or additional electronic device.

Preferably, the direct contact with the textile interface and/or the spaced apart arrangement is combined with a textile interface made of a mesh-like material to facilitate the arrangement and versatility of the apparatus, e.g., due to the homogenous structure. The mesh-like material furthermore facilitates the coating of the conducting wires and/or conducting pads, which is preferably provided by a silicon-based or other flexible coating material so as to improve the penetration of the first and second surface and facilitate the distribution of the coating material. In particular, such combination has the advantage that with a spaced apart arrangement encapsulation is facilitated at every region of the textile interface and fixation of the respective conducting pads may be improved. Thereby, the spaced apart arrangement also provides that the total consecutive area of the textile interface being encapsulated may be reduced, such that e.g. a more convenient wearing item may be provided that may better adapt to corresponding anatomical shapes and/or body movements.

According to one embodiment, the electronic device comprises a flexible printed circuit board. Preferably, the electronic device comprises a rigid printed circuit board. Accordingly, the electronic device may be configured as an electronic board or electronic card with an integrated circuitry provided by conducting paths and comprising electronic components that are in communication with the plurality of conducting pads so as to process the received sensor signals. The implementation of a flexible printed circuit board further increases the conformity of the apparatus to an anatomic region of a user and allows a deformation not only during use, but also during e.g. machine washing, such that external forces acting on the apparatus do not impair the mechanical stability and integrity of the apparatus. In addition, the flexibility facilitates the integration into e.g. a clothing item since the textile interface may be handled more easily, for example, when sewn into seams of a clothing item or garment.

Since the electronic device may be sized and dimensioned to cover only a minimal area of the textile interface and a height may be minimized by avoiding mechanical fasteners or plugging arrangements, a relatively small rigid printed circuit board may be used as part of the electronic device. Such rigid PCB further ensures that material contacts and bonds are maintained and unsoldering events are avoided, even during machine washing. Thereby, the integrity of the apparatus may be further increased.

Due to the full encapsulation of the sensitive electronic compartments, the apparatus may be integrated in a secure fashion into a variety of clothing items and may function independent of external influences. The apparatus may hence be implemented in a wearable fabric, wherein the sensor signals received from respective sensors coupled to a free end of respective conducting wires provide a monitoring of biometric parameters of the user. To further increase the ease of use and independent functioning of the apparatus, the apparatus is preferably configured as portable stand-alone apparatus, wherein the electronic device comprises an electrical energy storage device connected to a charging interface.

Thereby, a user does not require any additional devices and may record biometric parameter data without any additional wiring or power supply unit, i.e. as a fully wireless implementation. The electrical energy storage device, e.g. a rechargeable battery or cell, hence provides the energy required to receive and process the sensor signals into biometric parameter data, such that the user may be provided with an overview or monitoring of the current or actual status of its physiological status. While the electrical energy storage device (i.e. battery) is also coated with the coating material, i.e. is within the encapsulation to ensure a fluidic sealing, the charging interface may be provided at a region of the textile interface that is not covered by the coating material, such that the encapsulated area may be kept small and the charging interface is directly accessible.

Preferably, the charging interface is formed as two connecting interfaces, which are each connected to the electronic device via a conducting wire sewn into the textile interface. Thereby, opposite charging terminals may be provided that are securely attached to the textile interface via the conducting wires. Furthermore, this allows that the same method of attachment may be implemented in securing the charging interface, which not only facilitates the manufacturing, but also increases the structural integrity and stability of the apparatus.

To further facilitate the charging and to provide an intuitive coupling of a charging device with the charging interface, each connecting interface preferably comprises an attachment means, preferably a hook-and-loop attachment or snap fastener. Accordingly, each of the charging terminals may only be coupled to a terminal of the charging device having a matching configuration, such that a mismatch and potential damage to the electrical energy storage device may be avoided. The charging interface may also be formed, at least in part, by conductive yarn.

As described in the above in view of the conducting pads, the electronic device may also comprise at least one through hole, wherein at least one conducting wire extends through said through hole and secures the electronic device to the textile interface. Thereby, the electronic device may be easily attached to the textile interface, for example, by means of the fixation of integrated conducting pads and one or more holes at opposing ends of the electronic device. Furthermore, this avoids any relative movement between the conducting wires and the electronic device.

More preferably, a conducting wire connecting the electrical energy storage device to the charging interface may extend through the additional one or more holes, such that no separate holes and conducting wires are required for securing the electronic device to the textile interface. This further increases the structural integrity of the electronic device and reduces the number of conducting wires and the overall weight and rigidity of the apparatus.

Although each conducting wire may be comprised of a plurality of conducting fibers, e.g. conductive yarn, or thin metal threads which together define a longitudinal shape of the conducting wire, this may not significantly affect the circumference of the combined plurality of fibers. Accordingly, each conducting wire preferably comprises an essentially homogeneous outer surface and is formed of a plurality of conducting wires that are at least in part connected by material bonding. Such homogeneity may e.g. be considered as a core of the conducting wire with no protruding fibers in a radial direction and wherein the fibers arranged at an outer circumference are arranged essentially adjacently to each other, so as to minimize a space therebetween and provide an essentially smooth outer surface. Such smoothing may furthermore be increased by material bonding, which may be provided by means of local welding or soldering.

The apparatus may not only be configured to receive and process sensor signals, but preferably is also configured to process and store the biometric signals into multi-parameter biometrics data, wherein the apparatus may comprise a transmitter or transceiver for transmitting said data. Thereby, the monitoring may be provided by an external or remote device, which may give feedback to a user in the form of a visual representation, acoustic signal, and/or haptic or tactile feedback signal.

Preferably, the electronic device is configured to be wirelessly paired to a second device, in particular by reading out optical information attached to the apparatus on said second device. For example, the second device may be a smart phone or smart watch or computing device with a mobile interface, wherein the optical information may be present in the form of e.g. a scannable barcode or QR code. The second device then uses a camera or optical sensor for scanning said code and automatically provides a pairing between the second device and the electronic device of the apparatus, such that biometric data may be received from the apparatus. Thereby, the user, physician or medical personnel may quickly and easily couple or pair the apparatus with e.g. a wearable and/or mobile interface so as to monitor the biometric parameters on the second device, e.g. via Bluetooth, Zigbee, WLAN, NFC, RFID, or the like.

The invention further relates to a clothing item, preferably a t-shirt, comprising a wearable fabric and an apparatus as described in the above, wherein the textile interface of the apparatus is sewn into the wearable fabric, preferably at a region corresponding to the seams of the clothing item.

Accordingly, a fully wearable device is achieved, wherein the electronic device is embedded in the fabric of the clothing item. For example, the textile interface comprising the electronic components may be solidly sewn into a piece of tulle and comprise a coating comprising silicone, such that the apparatus comprises a high degree of flexibility, thereby facilitating the processing and integration of the apparatus in the clothing item.

This solution not only fully supports all body movements, but also allows multiple machine washes without requiring any modification or disassembly of the integrated apparatus. In other words, no plugging is needed during the use of the clothing item and the risk of detachment is significantly reduced due to the integrated structure and encapsulation, making the clothing item very easy-to-use. At the same time the presence of the electronic device is not apparent from the outside, thereby increasing the acceptance of a user.

Furthermore, by means of a charging interface, the electronic device may also be charged by means of a simple coupling to a power source, such that e.g. a battery does not need to be removed. For example, a charging may be provided by coupling a power source to charging terminals that are arranged on the textile interface outside of the encapsulation or may be performed by induction charging through the encapsulation material.

According to another aspect, a method of producing an apparatus for processing sensor signals is suggested, comprising the following steps:

providing an electronic device configured to process biometric signals of a user and a plurality of electrically conducting pads connected to the electronic device, wherein each pad is connected to a respective electrically conducting wire configured for contacting a sensor corresponding to an anatomic region of a user and for providing a sensor signal to the electronic device;

providing a textile interface for use in a wearable fabric having a first surface and a second surface and comprising a porosity providing a fluidic communication between the first surface and the second surface;

arranging and attaching the electronic device, the plurality of pads, and the plurality of conducting wires on the first surface of the textile interface; and

providing a coating material over at least the electronic device and the plurality of pads and extending from the first surface to the second surface so as to fluidically seal the electronic device and the plurality of pads.

According to a preferred embodiment, the biometric signals received from sensors are selected in the group of electrocardiogram (ECG), electroencephalogram (EEG), breathing pattern (including abdominal and costal patterns), lung impedance, body temperature, muscle activity or contraction, blood pressure and any other physiology signal.

The method hence allows a manufacturing of an apparatus that is fluidically sealed, thereby protecting the inner electronic components and, in particular, the electronic device. The coating hence replaces solid casings and plugging fixations, such that the electronic device forms a fully integrated feature of the textile interface and detachments of electronic components are essentially avoided.

The apparatus obtained by the method may generally correspond to the apparatus as described in the above, such that like features and technical advantages are not described again and it is instead referred to the description of the apparatus in the above.

To facilitate the arrangement and the attachment of the various components, the method may use a template printed on e.g. paper and using the desired dimensions of the apparatus. The textile interface may e.g. be at least semi-transparent and be arranged over the paper, wherein the paper and textile interface are cut along indicated lines that define the outer boundaries of the apparatus, which generally corresponds to the outer boundaries of the textile interface. The textile material may be folded around the paper material along a margin, such that the textile interface and paper template remain connected during further processing.

The electronic device, conducting pads, and conducting wires are then placed onto the first surface of the textile interface at the positions and according to the orientation indicated by the template and are attached to the textile interface. After attachment, the paper may be removed, e.g. by means of a forceps or tweezers. Subsequently, the electronic device and conducting pads are covered by a coating material, such that the coating material penetrates the textile interface and extends from the first surface to the second surface so as to fully cover said components at both sides, thereby forming an encapsulation. The coating may be provided by simple pouring or by spray coating, depending on the material and desired thickness of the coating.

The step of providing a coating material may include providing a first coating layer and a second coating layer over the first coating layer, wherein the coating material is preferably silicone-based. The first coating layer may e.g. be a relatively thin layer providing an initial cover of the electronic components, which facilitates the adherence of the second layer. The first and second coating layer may hence also be composed of different materials, but are preferably made of the same material so as to further facilitate the manufacturing. The second coating layer may then be applied after curing of the first coating layer and may comprise a greater thickness so as to provide a more homogeneous cover of the electronic components, which increases the structural stability and reduces the risk of detachment due to impact. In this regard silicone not only ensures that the electronic components are fluidically sealed, but also comprises a resilience upon deformation, such that forces acting upon the electronic components may be better distributed and damage to the electronic components is reduced or even avoided.

Alternatively, the step of providing a coating may include a step of injection molding with coating material according to well-known methods.

As described in the above, the textile interface is preferably a mesh material or tulle, wherein the conducting wire is attached to the textile interface by sewing using the conducting wire as a thread. The mesh material and, in particular, tulle provide a homogeneous surface, such that no further through holes are required and the existing holes may be used for sewing the conducting wires into the textile interface in the required orientation.

The conducting wires may be formed of conducting fibers or a lead wire that are used as a thread and may be inserted and used in a sewing machine, wherein the sewing needle performs the sewing. This may require that the sewing machine is initially configured according to the requirements of the attachment and the characteristics of the textile interface, e.g. the spacing between the respective holes (for example about 0.25 cm), such that further punctures and or wrinkles of the textile interface may be avoided. The lead wire may hence be provided on a role so as to provide a continuous segment that may traverse the textile interface and forms a conducting wire based on a plurality of fiber segments that define a core and a circumference based on the number of fibers. Any protruding fibers may furthermore be either removed or be welded or soldered by moving the welding or soldering iron along the outer circumference of the conducting wire, thereby achieving a material bonding of the plurality of fibers.

To facilitate the attachment of the conducting pads, each conducting pad preferably comprises at least two through holes, wherein the step of attaching the conducting pads to the textile interface preferably comprises sewing each contact pad into the into the textile interface using the respective conducting wire as a thread and by extending the conducting wire through the through holes. The through holes may be provided in advance, e.g. by stamping, punching, die-cutting or, preferably, by the sewing needle of the sewing machine. Alternatively, the holes may be formed during the sewing advancement, such that the sewing needle forms the hole while at the same time extending the conducting lead wire through the hole during the sewing movement.

The conducting pads may be integrated on the electronic device or may be in direct contact with the textile interface and/or be spaced apart from the electronic device. Accordingly, the conducting pads may e.g. be sewn into the textile interface independent of the electronic device so as to form a plurality of interconnected contact pads that together may form a control logic or “smart” textile interface. However, the conducting pads may also be integrated on the electronic device, for example, as integrated components on a (rigid) printed circuit board.

As for the conducting pads, the electronic device preferably comprises at least one through hole, wherein the step of attaching the electronic device to the textile interface preferably comprises sewing the electronic device using said through hole and using a conducting wire as a thread. Thereby, an attachment of the electronic device to the textile interface may be provided using the same sewing technique, such that the electronic device is integrated into the textile interface and separation is avoided without requiring e.g. plugging arrangements or mechanical fasteners or the like. The attachment is furthermore independent of the coating or encapsulation, such that the stress provided on said coating may be reduced.

The above method may advantageously be implemented in the production or manufacturing of producing a clothing item, preferably a t-shirt, comprising the above steps and by providing a wearable fabric, wherein the textile interface is sewed into the wearable fabric, preferably at a region corresponding to the seams of the piece of clothing. The textile interface, e.g. a flexible tulle material, carrying the electronic components may hence be folded into the seams of a garment at the required position to obtain sensor signals related to biometric parameters from a corresponding anatomic region when wearing the garment. The textile interface allows an easy integration into the wearable fabric, such that the wearable fabric may conceal a significant portion of the apparatus, thereby not significantly affecting the overall appearance of the garment.

### BRIEF DESCRIPTION OF THE DRAWINGS

The present disclosure will be more readily appreciated by reference to the following detailed description when being considered in connection with the accompanying drawings in which:

FIG. 1 is a schematic top view representation of an apparatus according to the invention having a textile interface prosthetic;

FIG. 2 is a schematic top view representation of an apparatus according to FIG. 1 with a particular type of textile interface and an alternative arrangement of conducting wires;

FIGS. 3 is a cross sectional view of the apparatus according to FIG. 2 along line A-A;

FIGS. 4 is a cross sectional view of the apparatus according to FIG. 3 implemented in a clothing item; and

FIGS. 5A and 5B show a schematic top view of an apparatus similar to the embodiment according to FIG. 1 with an alternative arrangement of the conducting wires.

### DETAILED DESCRIPTION OF PREFERRED EMBODIMENTS

In the following, the invention will be explained in more detail with reference to the accompanying figures. In the Figures, corresponding elements are denoted by identical reference numerals and repeated description thereof may be omitted in order to avoid redundancies.

In FIG. 1 a schematic depiction of an apparatus 10 according to the invention is shown in a top view. The apparatus 10 comprises a textile interface 18 having a first surface and a second surface, wherein only the first surface is visible from the top. The textile interface 18 serves as a substrate, such that various components may be held by the textile interface 18. The borders or boundaries of the textile interface 18 are indicated with a solid line, which during manufacturing may be printed on a paper template to indicate the required dimensions and shape. In such case, the textile interface 18 may be formed of an at least partially transparent material and placed onto the paper template indicating a predefined arrangement according to scale. The solid line is furthermore depicted with a scissors symbol to indicate that the textile interface is to be cut along this line. Furthermore, margins 32 indicated by the dashed line indicate the boundary of the template, such that the textile interface 18 may e.g. be folded into or over said margin 32, if required.

The textile interface 18 according to the embodiment is made of a flexible material that comprises a predefined porosity to allow a fluid communication between the first surface and second surface (not shown). However, the textile interface 18 itself is preferably not readily deformable, such that it provides a substrate having sufficient rigidity and structural integrity carry various components. Accordingly, an electronic device 12 is placed onto the textile interface 18, which is optionally configured as a printed circuit board with an integrated microprocessor and an integrated circuitry in the form of connecting conducting paths. The printed circuit board is optionally formed of a small-sized rigid printed circuit board, such that the integration of the electronic device 12 into the textile interface 18 does not adversely affect the flexibility of the textile interface 18, thus facilitating the handling and further processing of the textile interface 18.

The electronic device 12 is configured to process biometric signals of a user and may optionally be configured as a health monitor or as a part of a medical device. In order to process such signals, the electronic device 12 is connected with a plurality of integrated electrically conducting pads 14 that are each connected to a respective electrically conducting wire 16, as shown on the left of FIG. 1. The conducting wires 16 are hence linked to the conducting pads 14 and extend directly from said pads 14. Accordingly, when the textile interface 18 is brought into contact with an anatomic region of a user, e.g. when the textile interface 18 and the apparatus 10 as a whole is integrated in a clothing item, the conducting wires 16 may be coupled with respective sensors provided on the clothing item, e.g. by contacting a respective sensor with a free end of a respective conducting wire 16, such that electric signals may be received by the conducting wires 16 and transduced to the electronic device 12 via the conducting pads 14.

To facilitate that electric signals may be continuously obtained, the conducting wires 16 may furthermore comprise one or more curves or bent portions so as to provide a curved conducting wire 28 or bent conducting wire 30. This not only ensures an optimized contacting surface or matching to the anatomic region, but also ensures that the electric signals are received from a predefined region and furthermore exhibit a predefined spacing. It will be obvious that the number of conducting wires 16, 28, 30 and the number of pads 14 as well as the respective arrangements and orientations are merely exemplary and other arrangements and configurations may be implemented.

The apparatus 10 furthermore comprises a coating material 20, which is also indicated with a solid line and encloses the electronic device 12 and the conducting pads 14. The coating material 20 fully covers the electronic device 12 and the conducting pads 14 and furthermore covers a connecting region of the conducting wires 16, i.e. at an end extending from the conducting pads 14. This ensures that the conducting wires 16 may also at least partly be fixed by the coating material 20 and relative movement between the conducting wires 16 and the conducting pads 14 is prevented.

The coating material 20 hence forms an encapsulation for the sensitive electronic components of the apparatus 10. Although not shown in FIG. 1, the coating material 20 extends from the first or top surface to the second or bottom surface due to the porosity of the textile interface 18. Thereby, a full fluidic sealing is provided. In the embodiment, the coating material 20 is composed of a silicone material, which offers some degree of resilience and is easy to apply and process while ensuring that no liquids may penetrate the coating material 20 into the electronic device 12. The coating material 20 is depicted as a transparent material, however, other optical properties may be chosen, e.g. via material selection and/or coloring agents, such that the electronic components may also be concealed and/or the color may match the textile interface 18 and/or a wearable fabric into which the textile interface is integrated at a later stage.

The encapsulation provided by the coating material 20 hence results in a light weight yet robust apparatus 10, which omits the use of any mechanical fixations and casings, such that the apparatus 10 comprises a reduced thickness and does not require any plugging for attaching the electronic device 12 to the textile interface 18.

Although a variety of methods may be used to attach the conducting wires 16, the conducting pads 14 and the electronic device 12 to the textile interface 18, the embodiment according to FIG. 1 has conducting wires 16, 28, 30 that are sewn into the textile interface 18, wherein the thread is the conducting wire 16, 28, 30 and which traverses the first and second surface of the textile interface 18. Accordingly, a sewing pattern may be achieved using a conducting lead wire, so as to form respective conducting wires 16, 28, 30 formed of a plurality of conducting segments or fibers, e.g. conductive yarn. Using such arrangement and attachment, no further attachment means are required while at the same time this ensures that the electronic components of the apparatus 10 are securely attached to each other, such that any separation or detachment may be avoided. While the conducting wires 16, 28, 30 are intertwined with the textile interface 18, the conducting pads 14 and the electronic device 12 may comprise one or more through holes through which the conducting wires 16, 28, 30 extend, e.g. via a closed loop.

For providing a required electrical energy to the electronic device 12, the apparatus 10 may furthermore comprise e.g. a battery. Such battery (not shown) may be integrated in the electronic device 12, wherein a charging interface 22 arranged outside of the encapsulation may be connected to the battery via respective conducting wires. Accordingly, the battery may be charged using the first and second charging terminal 24, 26, which are connected to the battery via corresponding conducting plates or pads, indicated on the right of the electronic device 12 by the black rectangles.

Thereby, the apparatus 10 may function fully independently and may be easily integrated into e.g. a wearable fabric so as to provide a “smart” clothing item.

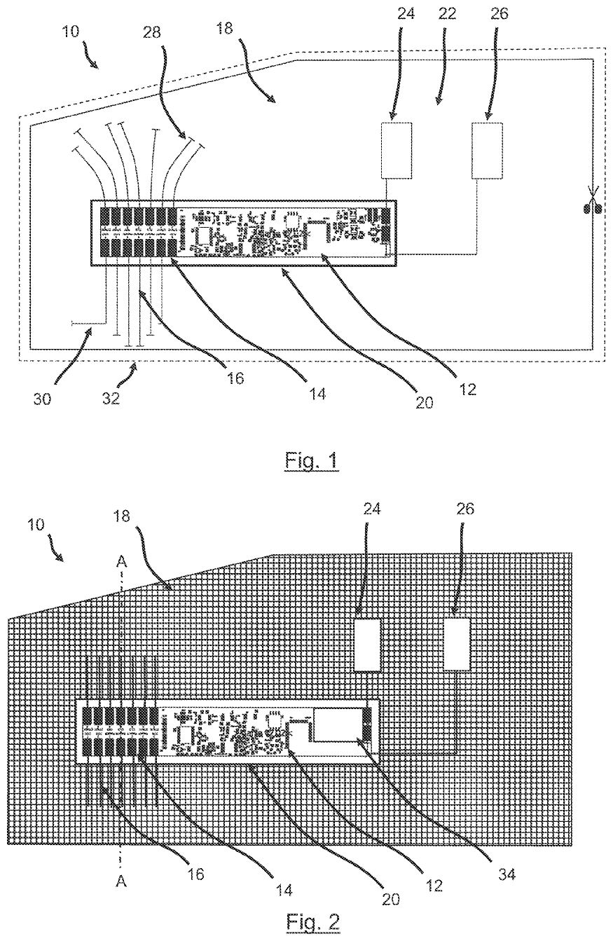

To further facilitate the encapsulation of the electronic components and to provide an easier handling of the textile interface 18, the textile interface 18 may be formed of a mesh material, such as tulle, as depicted in the embodiment according to FIG. 2. Accordingly, a flexible material is provided having a homogeneous structure with sufficient structural integrity to be used in clothing implementations. Tulle has the advantage that it increases the acceptance by a user and increases the breathability due to the continuous arrangement of holes. Furthermore, this significantly reduces effort when integrating the textile interface 10 in a wearable fabric of a clothing item since the tulle may be bent and folded as desired, depending on the clothing characteristics and boundaries. In addition, due to the plurality of holes in all directions no further holes or punctures in the textile interface 18 are required while at the same time the degree of flexibility of arranging and attaching the respective components is greatly enhanced.

In FIG. 2 a battery 34 is furthermore depicted as an integrated component of the electronic device 12. In addition, an alternative arrangement of the conducting wires 16 is provided. This provides a more compact design of the apparatus, which may be desirable if e.g. an equal spacing between the conducting wires 16 is required and no relative movements or curvatures of anatomic regions are expected in the area wherein the apparatus 10 is applied. However, it will be obvious that the such arrangement is not limiting and various alternative configurations are also envisaged.

A cross-sectional view of the apparatus 10 along the line A-A depicted in FIG. 2 is shown in FIG. 3. Here, a more detailed schematic view of the arrangement of the various components of the apparatus is provided. Accordingly, the conducting wires 16 extend from the conducting pads 14 in a single plane with the electronic device 12 and the textile interface 18, as also follows from the top view in FIGS. 1 and 2. Furthermore, it is shown that the electronic components are placed on the first surface 36 of the textile interface 18 while the coating material 20 extends from the first surface 36 through the textile interface 18 to the second surface 38 so as to form an encapsulation of the electronic device 12 and the conducting pads 14. In addition, the encapsulation also includes a connecting region of the conducting wires 16, i.e. at a portion where the conducting wires originate from the conducting pads 14.

The attachment of the conducting pads 14 and the electronic device 12 via the conducting wires 16 is not shown in detail, however, it may be envisaged that each of said components comprises one or more through holes, e.g. two or three holes, through which the conducting wire 16 extends.

Such attachment, i.e. a sewing attachment is schematically depicted by the traversing of the conducting wires 16 through the holes 40, so as to form an attachment and connection with the textile interface 18. Following essentially the same plane as the conducting pads 14, the textile interface 18, and the electronic device 12, the conducting wires 16 do not significantly protrude in a direction perpendicular to the first and second surface 36, 38 and hence form a smooth surface. Although the specific pattern may vary, it is generally contemplated that the sewing arrangement comprises both forward and backward stitches so as to ensure that the conducting wire 16 may not accidentally be loosened during prolonged use.

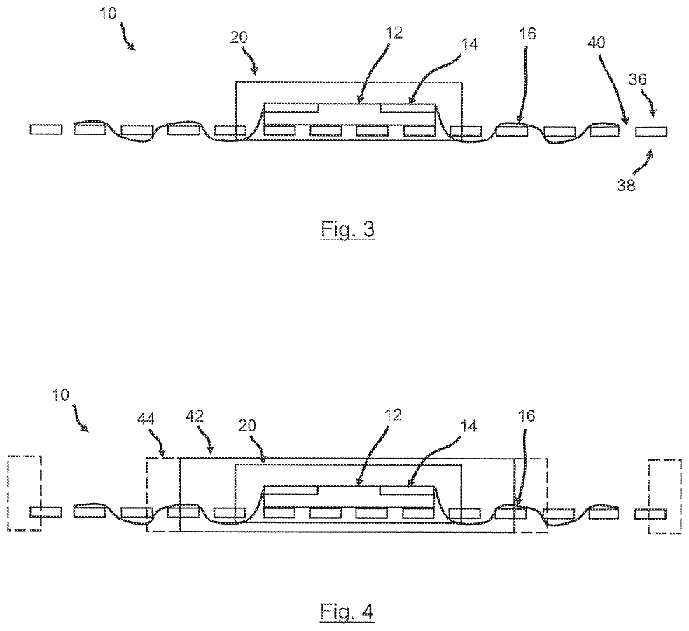

In FIG. 4 an embodiment is shown wherein the apparatus 10 according to FIG. 3 is sewn into a clothing item. Accordingly, the textile interface 18 is integrated in a wearable fabric 40 by sewing the textile interface 18 into seams 42 of the wearable fabric 40, which are indicated by the dashed lines. This results in an embedded electronic device 12 in a garment or clothing piece, wherein a portion of the conducting wires 16 may not be covered by either the textile interface 18 or the wearable fabric 40 and forms a free end, which may be exposed, brought into contact with, or coupled to a sensor arranged at the wearable fabric 40 and corresponding to an anatomic region of a user, when wearing the clothing item. Thereby, the conducting wires 16 may obtain signals, e.g. electric signals related to physiological parameters, which are transduced to the conducting pads 14 and are received by the electronic device 12, wherein the electronic device 12 processes said signals and determines one or more biometric parameter values therefrom.

The determined biometric parameters and corresponding values may then e.g. be transmitted to a coupled second device via a transmitter arranged on the electronic device 12 to provide a monitoring of a physiological status or health status of the user. Alternatively, or in addition, the electronic device 12 may comprise e.g. a signal outputting device such as an electromechanical transducer or buzzer to alarm a user when the determined parameter value exceeds a physiological boundary. Thereby, various arrangements may be provided to process the sensor signals and provide a feedback to the user or e.g. a physician, physiotherapist, or sport coach.

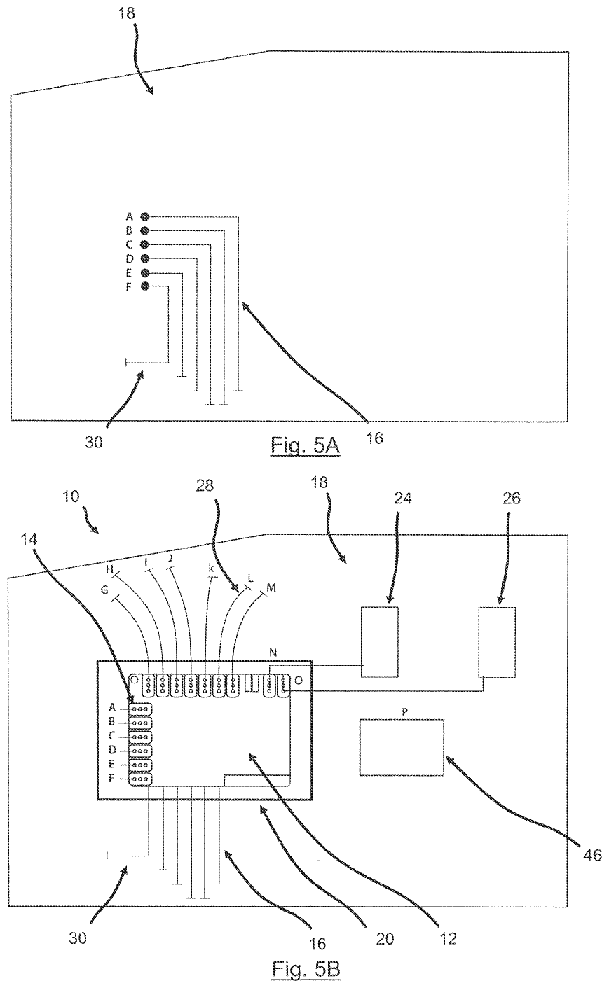

FIGS. 5A and 5B show a schematic top view of an apparatus 10 similar to the embodiment according to FIG. 1 with an alternative arrangement of the conducting wires 16. In this embodiment, the conducting wires 16 are arranged either below the electronic device 12 (A to F) or on top of the electronic device 12 (G to M), such that the conducting wires 16 arranged underneath the electronic device 12, i.e. conducting wires A-F, are to be attached to the textile interface 18 before placing the electronic device 12 on the textile interface 18. Accordingly, FIG. 5A depicts the textile interface 18, wherein the conducting wires A-F are securely attached to the textile interface 18 using a sewing technique, such that the conducting wires A-F extend in an essentially straight fashion as indicated with the reference numeral 16 or in a bent fashion, as indicated by the reference numeral 32. The thick dot indicates both the starting point and end point of the sewing thread or conducting wire.

From each respective starting and end point, a free end extends from the textile interface 18, which is intended for attachment to the corresponding conducting pad 14, as shown in FIG. 5B. Accordingly, the free ends are pulled away from the textile interface 18 and are held towards the left of the textile interface 18 prior to placing the electronic device 12 on the textile interface 18 and the conducting wires A-F. Thereby, a short-circuiting is prevented and the free ends of the respective conducting wires A-F may be attached to the conducting pads 14 via the through holes of the conducting pads 14 and by using the same sewing technique.

After attachment of the conducting wires A-F to the conducting pads 14, the conducting wires G-M are attached to the respective conducting pads 14 and are attached to the textile interface 18 by a corresponding pattern, providing curved conducting wires 28. The first and second charging terminals 24, 26 may then be connected to the textile interface 18 and the electronic device 12 and the coating material 20 may then be applied to the textile interface 18 and the electronic device 12 as described in the above.

Furthermore, an identification tag 46 may be attached to the textile interface 18, which allows an identification of the electronic device 12 after applying the coating material 20 and/or implementation in a clothing item. Such identification may provide a coupling with an external device and/or may perform a calibration or installation via wireless transmission, such that the apparatus may form an interface for a user providing biometric parameter data according to a particular configuration. For example, the identification tag 46 may be a scannable object such as a QR-coded label, which may be read by an optical reader, e.g. integrated in a mobile terminal or portable device so as to provide a coupling between the apparatus and the portable device.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 Apparatus
12 Electronic device
14 Conducting pad
16 Conducting wire
18 Textile interface
20 Coating material
22 Charging interface
24 First charging terminal
26 Second charging terminal
28 Curved conducting wire
30 Bent conducting wire
32 Margin
34 Battery
36 First surface
38 Second surface
40 Through hole
42 Wearable fabric
44 Seam
46 Identification tag

1. An apparatus for processing sensor signals, comprising:
an electronic device configured to process biometric signals of a user;
a plurality of electrically conducting pads connected to the electronic device, wherein each pad is connected to a respective electrically conducting wire configured for contacting a sensor corresponding to an anatomic region of a user and for providing a sensor signal to the electronic device; and
a textile interface for use in a wearable fabric having a first surface and a second surface and comprising a porosity providing a fluidic communication between the first surface and the second surface, wherein the electronic device, the plurality of pads, and the plurality of conducting wires are arranged on the first surface of the textile interface and are attached to the textile interface;
wherein the apparatus further comprises a coating material covering at least the electronic device and the plurality of pads and extending from the first surface to the second surface so as to fluidically seal the electronic device and the plurality of pads.

2. The apparatus according to claim 1, wherein the coating material is a flexible material, preferably comprising silicone.

3. The apparatus according to claim 1, wherein the coating material is molded over at least the electronic device and the plurality of pads.

4. The apparatus according to claim 1, wherein the porosity of the textile interface is provided by a plurality of holes at essentially equal spacing from each other.

5. The apparatus according to claim 4, wherein the textile interface is a mesh material, preferably tulle.

6. The apparatus according to claim 4, wherein each conducting wire is sewn into the textile interface, wherein each conducting wire comprises a thread traversing the first and second surface of the textile interface.

7. The apparatus according to claim 1, wherein the coating material is a flexible material and wherein the porosity of the textile interface is provided by a plurality of holes at essentially equal spacing from each other, said textile interface being a mesh material, and wherein each conducting wire is sewn into the textile interface, wherein each conducting wire comprises a thread traversing the first and second surface of the textile interface.

8. The apparatus according to claim 7, wherein the coating material comprises silicone or is silicone-based and/or wherein the textile interface is made of tulle.

9. The apparatus according to claim 6, wherein each of the conducting pads is secured to the textile interface by a respective one of the plurality of conducting wires.

10. The apparatus according to claim 9, wherein each conducting pad comprises at least one through hole and is sewn into the textile interface by the respective conducting wire extending through the at least one through hole.

11. The apparatus according to claim 10, wherein each conducting pad comprises two or three through holes.

12. The apparatus according to claim 1, wherein each conducting wire extends from the respective pad in a plane defined by the electronic device and the plurality of conducting pads.

13. The apparatus according to claim 1, wherein each conducting wire comprises an essentially straight portion, a curved portion, and/or a bent portion.

14. The apparatus according to claim 1, wherein the coating material covers a portion of each conducting wire connecting the conducting wire to the conducting pad.

15. The apparatus according to claim 1, wherein the conducting pads are integrated on the electronic device.

16. The apparatus according to claim 1, wherein the conducting pads are in direct contact with the textile interface and/or are spaced apart from the electronic device.

17. The apparatus according to claim 16, wherein the conducting pads are in direct contact with the textile interface and are spaced apart from the electronic device.

18. The apparatus according to claim 17, wherein the coating material is a flexible material, wherein the porosity of the textile interface is provided by a plurality of holes at essentially equal spacing from each other, said textile interface being a mesh material, and wherein each conducting wire is sewn into the textile interface, wherein each conducting wire comprises a thread traversing the first and second surface of the textile interface and wherein the conducting pads are in direct contact with the textile interface and are spaced apart from the electronic device.

19. The apparatus according to claim 1, wherein the electronic device comprises a rigid printed circuit board.

20. The apparatus according to claim 1, wherein the apparatus is configured as a portable stand-alone apparatus and wherein the electronic device comprises an electrically energy storage device connected to a charging interface.

21. The apparatus according to claim 20, wherein the charging interface is formed as two connecting interfaces, each connected to the electronic device via a conducting wire sewn into the textile interface.

22. The apparatus according to claim 21, wherein each connecting interface comprises an attachment means, preferably a hook-and-loop attachment or snap fastener.

23. The apparatus according to claim 1, wherein the electronic device comprises at least one through hole and wherein at least one conducting wire extends through said through hole and secures the electronic device to the textile interface.

24. The apparatus according to claim 1, wherein each conducting wire comprises an essentially homogeneous outer surface and is formed of a plurality of conducting wires that are at least in part connected by material bonding.

25. The apparatus according to claim 1, wherein the electronic device is configured to process and store the biometric signals into multi-parameter biometrics data and comprises a transmitter or transceiver for transmitting said data.

26. The apparatus according to claim 25, wherein the electronic device is configured to be wirelessly paired to a second device, preferably by reading out optical information attached to the apparatus on said second device.

27. A clothing item, preferably a t-shirt, comprising a wearable fabric and an apparatus according to claim 1, wherein the textile interface of the apparatus is sewn into the wearable fabric, preferably at a region corresponding to the seams of the clothing item.

28. A method of producing an apparatus for processing sensor signals, comprising the steps of:
providing an electronic device configured to process biometric signals of a user, and a plurality of electrically conducting pads connected to the electronic device, wherein each pad is connected to a respective electrically conducting wire configured for contacting an anatomic region of a user and for providing a sensor signal to the electronic device;
providing a textile interface for use in a wearable fabric having a first surface and a second surface and comprising a porosity providing a fluidic communication between the first surface and the second surface;
arranging and attaching the electronic device, the plurality of pads, and the plurality of conducting wires on the first surface of the textile interface; and
providing a coating material over at least the electronic device and the plurality of pads and extending from the first surface to the second surface so as to fluidically seal the electronic device and the plurality of pads.

29. The method according to claim 28, wherein the step of providing a coating material includes injection molding with coating material, wherein the coating material is preferably an elastomer or more preferably silicone-based.

30. The method according to claim 28, wherein the textile is a mesh material or tulle and wherein the conducting wire is attached to the textile interface by sewing using the conducting wire as a thread.

31. The method according to claim 28, wherein the method provides an apparatus according to claim 1.

32. The method according to claim 30, wherein each conducting pad comprises at least two through holes and wherein the step of attaching the conducting pads to the textile interface comprises sewing each contact pad into the into the textile interface using the respective conducting wire as a thread and by extending the conducting wire through the through holes.

33. The method according claim 32, wherein the conducting pads are integrated on the electronic device or wherein the conducting pads are in direct contact with the textile interface and/or are spaced apart from the electronic device.

34. The method according to claim 29, wherein the electronic device comprises at least one through hole and wherein the step of attaching the electronic device to the textile interface comprises sewing the electronic device using said through hole and using a conducting wire as a thread.

35. The method of producing a piece of clothing, preferably a t-shirt, comprising the steps of the method according to claim 28 and providing a wearable fabric and sewing the textile interface into the wearable fabric, preferably at a region corresponding to the seams of the piece of clothing.
